(19) United States
(12) Patent Application Publication
(10) Pub. No.: US 2003/0092089 A1
(43) Pub. Date: May 15, 2003

(54) Method for diagnosing multiple sclerosis and an assay therefore

(76) Inventors: Mario Anthony Moscarello, Toronto (CA); Andrea Chamczuk, Toronto (CA)

Correspondence Address: MCHALE & SLAVIN, 4440 PGA BLVD, SUITE 402, PALM BEACH GARDENS, FL, 33410

(21) Appl. No.: 09/992,174

(22) Filed: Nov. 14, 2001

(51) Int. Cl. G01N 33/53; G01N 33/537; G01N 33/543

(52) U.S. Cl. 435/7.92

(57) ABSTRACT

This invention is directed toward a serum/plasma assay for the diagnosis and subsequent monitoring of patients with multiple sclerosis (MS). Assay performance characteristics indicate that the assay is accurate and repeatable. Using blood from patients with clinically definite multiple sclerosis, a clinical sensitivity of 77% and a specificity of 95% has been achieved through the measurement of circulating myelin basic protein autoantibodies. The assay provides a simple, rapid, and minimally invasive tool for the diagnosis and monitoring of progression of MS.

### FIELD OF THE INVENTION

The present invention relates to a method for the detection of biological materials relating to the prediction, diagnosis, or monitoring of progression of an autoimmune disease, utilizing an assay system developed to measure levels of biochemical markers involved in autoimmune disease. More specifically, this invention relates to an assay for detecting myelin basic protein (MBP) autoantibodies alone, and alternatively in conjunction with the measurement of other biochemical markers associated with multiple sclerosis (MS) and related diseases. Most specifically, the present invention is directed toward a process for initially diagnosing MS, and toward the para-clinical work-up and routine monitoring of MS patients as to disease progression.

### BACKGROUND OF THE INVENTION

Multiple Sclerosis (MS) is a chronic inflammatory demyelinating disease of the human central nervous system (CNS). Pathologically, the disease presents focal areas of myelin destruction, known as plaques or lesions (Vollmer, 1999). Myelin is present as a multilamellar sheath formed by membranous extensions of oligodendrocytes within the CNS (Peters, 1960a; b). Its ability to insulate axons and facilitate rapid nerve conduction is attested to by its high lipid content (70% of the total weight). With the focal deterioration of the insulating myelin sheath, the lesions result in decreased conduction velocity through naked regions of the axons affected. Such primary demyelination manifests as the symptoms of MS. Such symptoms include motor weakness in one or more limbs, optic neuritis, diplopia, parasthesia, fatigue, and eventually paralysis and early morbidity (Pender, 1995). Although the aetiology is unknown, geographic, genetic, and immune factors, acting coordinately, often determine disease onset and severity. Given the early onset of the disease (with diagnosis in children increasing in frequency), usually in the third or fourth decade of life, MS is one of the leading causes of neurological impairment in young adults. The precise mechanism by which demyelinating lesions are formed in MS is not known, however the most promising theory is that demyelination occurs as a result of aberrant immunology resulting in immune attack of myelin. The association between histocompatibility phenotype and susceptibility in MS has immunological implications. Furthermore, clinical features in MS, such as the presumed long latent period, the chronic nature of the disease and the pattern of acute attacks followed by remission are suggestive of an immunologically mediated disease. Pathologically, MS lesions are marked by an infiltration of numerous immune cells, especially T-cell lymphocytes and macrophages (Pender, 1995). Histological and immunological similarities between MS and the classic animal model of the disease, experimental allergic encephalomyelitis (EAE), have suggested that the disease may be T-cell mediated (Esiri, 1991). However, evidence of humoral immunity in MS is also abundant as reflected by the presence of B-cells and plasma cells within MS lesions and the elevated levels of cerebrospinal fluid (CSF) immunoglobulin G (IgG) in MS patients. Although the autoantigen responsible for MS has not been conclusively identified, myelin basic protein (MBP) has been proposed as a candidate autoantigen.

MBP is a cationic membrane-associated protein found in myelin in the CNS, in which it accounts for approximately 35% of the total myelin protein. In humans, there are four main isoforms generated by alternative splicing of a single transcript: 21.5 kDa, 18.5 kDa, 17.2 kDa and the 14 kDa isoforms (Kamholz, et al., 1986; 1988). The 18.5 kDa isoform, in which exon 2 is spliced out, is 170 amino acids in length (Carnegie, 1971) and is the most prominent isoform in mature human myelin (Moscarello, 1997; Schmidt, 1999). After the identification of MBP as the antigen associated with EAE, antibodies against MBP were detected in the CSF of MS patients and the antibody levels correlated with active MS disease status

In addition to MBP, protein markers worthy of investigation regarding MS autoantigens are:

1. Proteolipid protein (PLP) is a 30 kDa hydrophobic protein which constitutes approximately 50% of the total myelin protein. PLP is expressed in CNS myelin and is found in the myelin membrane (Lees and Brostoff, 1984).

2. S-100B (bb homodimer) is a 21 kDa acidic calcium binding protein expressed primarily in astrocytes, oligodendrocytes (Kimura et al., 1986; Richter-Landsberg and Heinrich, 1995) and Schwann cells, as well as other cells and tissues including adipose tissue, skeletal muscle, the retina, salivary glands, and immune cells (Zimmer et al, 1995).

3. The expression of neuron specific enolase (NSE) in oligodendroglial cells is associated with the period during which myelination takes place. NSE has been proposed as a biochemical marker for both neuronal tumors (glioblastoma, astrocytoma, and Schwannoma) as well as small cell lung carcinoma (Jorgensen, 1999), and NSE can be reliably detected in reactive astrocytes after brain injury (Lin et al., 1994). Although NSE has not been suggested as a marker in MS, it deserves serious consideration.

4. Thrombomodulin (Tm) is a 75 kDa endothelial cell surface transmembrane glycoprotein which functions to indirectly activate protein C, by binding to thrombin and altering its procoagulant activity (Dittman and Majerus, 1990). Thrombomodulin is thought to be released into the circulation when the endothelial cells are damaged (Tsukada et al, 1995). Therefore, damage to endothelial cells precedes thrombomodulin release into the circulation. Presumably, damage to the endothelial cells forming the blood-brain barrier (BBB) releases Tm into the CSF and is subsequently transported into the peripheral blood. Therefore, Tm may be released prior to the onset of clinical symptoms of relapse.

Traditionally, a good biochemical marker for any disease is characterized by a high clinical sensitivity and specificity for only the disease of interest. In the case of MS, numerous biochemical markers, including MBP, NSE, S-100B, and PLP autoantibodies, MBP, and thrombomodulin have been examined. Although these possible diagnostic measures have shown some extent of non-specificity for MS and variable clinical sensitivities, ranging from 0-50%, were observed, these biochemical markers have shown promise as diagnostic and prognostic tools attested by the fact that clinical sensitivities generally increased in patients experiencing active disease. This observation is especially characteristic of MBP autoantibody measurement in the CSF of MS patients.

Specific clinical signs and the temporal pattern of their presentation in the individual MS patient indicate the clinical course of the disease. Each disease course is remarkably different from the other, although the precise definition of the forms of the disease remains a constant source of debate and undergoes continuous revision. To summarize, the majority of cases of MS initially run a relapsing and remitting course (Matthews, 1991). This form of the disease is characterized by alternating phases of exacerbation/relapse (acute attack of neurological deficit) of symptoms and subsequent recovery with little or no residual deficits (Stinissen et al, 1997; Thompson et al, 1997). The duration of relapse ranges from a period of hours to months before any remission of the symptoms is observed. In many cases, patients first experience some degree of optic neuritis. Improvement/recovery after the first attack is sufficient to identify remitting disease (Vollmer, 1999).

On average, the proportion of patients with relapsing-remitting MS (RRMS) is 85% (Vollmer, 1999). RRMS presents with a characteristic alternation of phases between an acute attack of neurological deficit (relapse) and a period of recovery (stable). Patients older than 40 usually have the progressive form (Vollmer, 1999) of the disease. Secondary-progressive MS (SPMS) is characterized by significant neurological deficits that increase over time with relapses (Thompson et al, 1997). Many patients with RRMS progress to SPMS on average ten to fifteen years after the initial symptoms present, although some older patients (over 40 years at onset) experience the secondary progressive disease course from the outset.

Primary-progressive MS (PPMS) is less common and is characterized by patients with a slow and steady progression of impairments from onset of presentation without distinct attacks (relapses) (Al-Omaishi et al, 1999). Rarely, a more malignant form of MS occurs and is called the Marburg's variant. Marburg's disease is characterized by an acute fulminant monophasic course resulting in death three weeks to six months after the onset of initial symptoms (Lassman et al., 1981). Conversely, some MS patients can remain asymptomatic for 15 years or longer and are considered to follow a “benign” disease course. Disease course is variable between individual MS patients resulting in a continuum of severity which reflects an equally variable pathology and possibly aetiology.

Studies on the pathology of the demyelinating lesions in MS have led to the identification of numerous key players in the multifactorial pathogenesis of the disease. The release of components of the myelin sheath, evidence of blood-brain barrier (BBB) damage, astrocytic proliferation early in the formation of the plaque, gliosis, as well as infiltration of macrophages and B-cells within the lesion, and CD8⁺ T-cells surrounding the lesions are consistent features in MS pathology. These pathological characteristics facilitate both the identification of contributory factors in MS and provide clues as to which biochemical markers might offer the most utility in the diagnosis and prognostic monitoring of MS.

Although the CNS is usually an immune privileged site, the BBB is damaged during acute inflammation in the CNS, as serum proteins have been found throughout the lesions (Brosnan and Raine, 1996), resulting in novel access of lymphocytes to myelin and myelin components. While MBP autoantibody testing may be more useful in diagnosing MS, the measurement of MBP itself may indicate activity of lesions which would possibly precede clinical symptoms of relapse and may suggest immunodominant epitopes if the MBP material can be identified.

The diagnosis of MS presently relies heavily on clinical examination and neuroimaging techniques. Due to the heterogenous nature of the disease, accurate and rapid diagnosis is not only made difficult but is very costly and often quite painful, especially when the differential diagnosis includes diseases such as Behcet's disease, sarcoidosis with CNS involvement and monophasic demyelinating disease, including optic neuritis and acute disseminated encephalomyelitis (ADEM). ADEM is especially important in the differential diagnosis of MS in children.

Although a clinical sensitivity of ˜90% can be achieved using MBP autoantibody levels in the CSF, this method is unsuitable as a diagnostic tool for several practical reasons. CSF is extracted through lumbar puncture (LP) which is an invasive technique. Besides being uncomfortable to the patient, complications associated with LPs include prolonged headaches post-procedure, a risk of infection, and lengthy time duration for both patient and physician since a typical lumbar puncture often requires the patient's presence for several hours. Furthermore, the performance of LPs are reserved for experienced neurologists.

What has, until now, been lacking in the art, is a simple assay in which MBP autoantibodies are measured in the blood or in a blood product of MS patients or suspected MS patients. Unfortunately, no useful method currently exists for MS patients to monitor their disease status. Venipuncture has much less associated complications than LP, is usually performed as a standard procedure in any initial diagnostic work-up, and may be performed by individuals without any neurological background. In addition, the complications surrounding acid dissociation, as is required at present, would not be necessary. Since blood sampling can be performed frequently, MBP autoantibodies may demonstrate prognostic utility and predict disease progression. What is therefore needed is a reliable immunoassay which can be easily commercialized, could be integrated into the diagnostic work-up in MS, and may facilitate the monitoring of patients, including those involved in novel MS therapeutic trials.

With the recommendation for earlier treatment in MS, earlier and reliable diagnostic measures are of paramount importance. Since a disease course featuring spontaneous remissions make any treatment regimen problematic, tailoring treatments to individual sub-classifications of disease course, i.e., relapsing-remitting, primary-progressive, secondary-progressive, represent a more effective treatment method. Though the average duration of life is 10 to 20 years following onset, in actuality, many patients live longer. Some patients have frequent attacks and are rapidly incapacitated, while others have remissions that last as long as 25 years. Drugs such as interferon beta-1b (IFNβ-1b) are more effective in RRMS than SPMS. At present, clinical stratification of a patient is generally achieved through the patient's history. Only in PPMS (there is more spinal cord lesion involvement) is MRI utilized. Inclusion criteria for almost all drug trials include specified disease course.

Thus, if a minimally invasive technique involving at least one reliable biochemical marker indicative of disease activity, as well as disease course, could be employed not only for the diagnosis of MS and the prognostic monitoring of the disease for clinical trial, but also as a means of determining disease sub-classification for patient study inclusion, a long felt need would be satisfied.

### DESCRIPTION OF THE PRIOR ART

In a detailed and extensive study for determining the specificity of MBP autoantibody response in the CSF, Warren and Catz (Eur. Neurol., 42 (1999), 95-104) determined that 98% of other non-MS controls did not exhibit elevated anti-MBP antibodies. Warren and Catz further reported that 90-95% of MS patients with active disease had elevated levels of MBP autoantibodies in the CSF. Despite the work conducted using the CSF, the presence of MBP autoantibodies in the systemic circulation has not been conclusively determined. Previous work has favored measurement in the CSF and numerous investigators have reported limited success with their detection in serum. Experiments using serum samples have been plagued by problems associated with high background interference using a variety of assay methods. This is likely the result of insufficient optimization of assays for the enhanced sensitivity required for MBP autoantibody detection at low titers in a matrix containing so many other proteins. Furthermore, the lack of consistent methodology between investigators may account for the apparent discrepancy between their collective results. To illustrate this point, Reindl and colleagues (Brain, 122 (1999), 2047-2056) investigated the detection of anti-myelin/oligodendrocyte glycoprotein (anti-MOG) and anti-MBP IgG in the sera and CSF of MS patients. Using a Western blotting technique, positive detection of anti-MBP IgG was achieved in 28% of the total MS population studied. Since the nature of MBP autoantibodies in serum is of relatively low titer, at an equivalent polyclonal dilution, especially compounded with the issue of high background as seen through the Westerns performed, it is now understandable that patients could not be detected as positive.

Several investigators have established that autoantibodies against several myelin and non-myelin proteins can be detected at various frequencies in the CSF of MS patients. At the forefront of this work is the detection of MBP autoantibodies in the CSF of MS patients. While Panitch (Arch. of Neurol., 37 (1980), 206-209) reports a clinical sensitivity of 81% in his MS patients, Warren and Catz (Annals of Neurol., 20, 1 (1986), 20-25) observed a sensitivity of 55% when free (no acid dissociation) levels were assayed and a clinical sensitivity of 56% when bound fractions were measured.

Previous work performed in the CSF demonstrated that MBP was reliable indicator of relapse, correlated well with the number of gadolinium-enhanced lesions on MRI, EDSS scores, intrathecal IgM synthesis (Lamers et al, 1998), and levels of CSF MBP correlated well with CSF free anti-MBP levels (Warren and Catz, 1987).

The most consistent examination of MBP autoantibodies in the CSF has been performed by Warren and Catz (1986-present). Their extensive work rests on the supposition that MBP autoantibodies are frequently complexed with MBP and in order to detect the autoantibodies, acid dissociation with 1N acetic acid for release is required. It should be noted that the diagnostic utility of MBP autoantibodies rests in this domain and that their diagnostic success lies in studying patients defined as having active disease (Warren and Catz, 1986), that is patients in relapse. Throughout their work, Warren and Catz have utilized acid dissociation of CSF samples to break MBP-autoantibody complexes, and their results are reported in sub-classified groups of CSF autoantibody responses. These responses are free (non-dissociated, % bound radioactivity), total (dissociated), bound (total minus free) and a final free/bound ratio. All patients experiencing acute exacerbations demonstrated elevated levels of free MBP autoantibodies. Patients in the progressive form of the disease had elevated bound levels of anti-MBP. Elevated levels of either free or bound anti-MBP were not detected in patients with clinically inactive disease. This group reasoned that long-term and/or repeated MBP release into the systemic circulation is likely to enhance the immune memory for anti-MBP synthesis. In further support of the increased frequency of anti-MBP responses, patients with optic neuritis (ON) were examined for their autoantibody response to MBP. Warren and Catz found that ON patients demonstrated elevated levels of free MBP autoantibodies by radioimmunoassay (RIA) (Warren and Catz, 1994). It appears that anti-MBP is associated with active phases of disease in MS especially since MS patients frequently present initially with attacks of ON. However, given the requirement of acid dissociation before testing, this RIA procedure would not provide a simple method of testing.

Although the precise mechanism by which MBP is released from the myelin membrane has not been entirely worked out, measurements by several groups have revealed that MBP in the CSF, when measured by RIA, can be a reliable indicator of disease activity (Whitaker, 1977; Whitaker and Herman, 1988). Patients exhibited the highest levels of CSF MBP if they were experiencing acute exacerbation/relapse (Whitaker, 1977; Thompson et al, 1985; Warren et al, 1985; Frequin et al, 1992 and Ohta et al, 2000), whereas the majority of clinically stable/remitting patients had levels of CSF MBP comparable to that observed in other neurological disease controls. Furthermore, in patients in clinical remission who had significantly elevated MBP levels, sub-clinical demyelination was also detected by MRI (Thompson et al, 1985) which supports the role of MBP in CSF as a marker for myelin destruction. CSF MBP levels were reduced to normal levels when previously relapsing patients were treated with high doses of methylprednisolone (Frequin et al., 1992). This reduction in CSF MBP correlated well with a decrease in intrathecal IgM synthesis, which is also known to increase during relapse. Taken together, MBP, having also significantly correlated with EDSS score during relapse and the number of gadolinium-enhanced lesions on MRI, has shown promise as an indicator of MS pathology (Thompson et al., 1985; Frequin et al, 1992). Unfortunately, the clinical utility of MBP in the serum of MS patients has not been pursued because the extent of MBP peptide generation by circulating proteases has not been determined, resulting in the lack of a sensitive assay which specifically measures circulating MBP/MBP-peptides. Measurement of MBP in serum would perhaps provide a simple means of monitoring the progression of disease in an individual patient. Little research has been reported for the clinical specificity of the MBP autoantibody response in serum, presumably because many investigators have had such limited success in their detection in MS patients.

The Schumacher criteria for the diagnosis of MS is based solely on clinical examination and history (a more detailed discussion to follow). With the advent of neuroimaging techniques in the 1970's and advances in electrophysiology in the 1980's, demyelinating lesions suggestive of MS became visually apparent in some cases even prior to their clinical manifestations. In addition, abnormalities in CSF immunoreactivity, such as the presence of oligoclonal bands, increased IgG synthesis, and elevated IgG, became useful diagnostic criteria, but none were definitive. The existing clinical criteria did not accommodate the new laboratory techniques for diagnosing MS. In 1983, Poser and his colleagues published a new set of diagnostic guidelines which added the demonstration of para-clinical lesions (i.e. lesions visualized by MRI or CT scan or evoked-potential testing) as part of the clinically definite MS diagnostic criteria, as well as a new diagnostic category of laboratory-supported definite MS which required the detection of oligoclonal bands in the CSF or increased CNS synthesis of IgG, as measured by the CSF IgG index (CSF IgG Index is determined by the ratio CSF/serum IgG: CSF/serum albumin)(Link, 1991).

For prognostic purposes, unambiguous diagnostic criteria for MS is of extreme importance for the patient. At the present time, the definitive diagnosis of MS is often times a lengthy process which renders the patient anxiously uncertain for days or months regarding the diagnosis and prognosis. In addition, defining a population of patients with “probable or possible” MS aids in the prospective evaluation of novel diagnostic tests for the disease (Poser et al., 1983) is often problematic. The clinical trials for testing novel MS therapies are now multi-centered and require a minimization of the subjectivity involved in the clinically-based diagnostic work-up performed by the examining neurologist. Resolution of these issues could be achieved if a simple, rapid, and clinically sensitive, diagnostic test was available to both family physicians and neurologists alike.

U.S. Pat. No. 5,747,274 to Jackowski discloses a variety of assay types, as well as various assay formats and automated analyzer apparatus.

U.S. Pat. No. 5,645,997 issued to Kline et al. describes an assay for detecting antigens associated with multiple sclerosis utilizing hybridomas to produce monoclonal antibodies specific for MS-associated antigens. Peripheral blood lymphocytes (PBL) are isolated from a blood sample, and then processed to extract the MS-associated mitogens from the whole PBLs, if they are present. Kline et al. also teaches the administration of an effective dose of MS-associated antigens or a fraction thereof or its associated nucleotide sequences or a fraction thereof.

U.S. Pat. No. 5,998,150 issued to Whitaker et al. provides a method for determining the status of an MS patient participating in an IFNβ-1b trial, by measuring levels of urinary myelin basic protein-like material in the patient for predicting failure of remission or presence of a progressive phase in MS. The measurement by radioimmunoassay, preferably a double-antibody radioimmunoassay, is said to correlate with the number of lesions or total area of lesions in an MS patient.

What is lacking in the art is a rapid immunoassay, sensitive in serum, to detect levels of markers involved in autoimmune diseases, specifically MS, wherein results are definitive and the requirements of the patient are simple. It is the intention of the present invention to offer an assay format which can be used as a rapid manual test to be administered at the point-of-care at any location.

### SUMMARY OF THE INVENTION

The present invention relates to the detection of biomolecules linked to multiple sclerosis, along with methods to assess severity of disease with routine monitoring. The presence, in excess, of autoantibodies to myelin basic protein in blood has now been shown to be indicative of MS.

The present invention contemplates assembly of a kit for diagnosis of MS and monitoring of previously diagnosed MS patients, thus providing simple and definitive results, on a regular basis, which may assist in predicting an upcoming acute attack. Consequently, preventive therapy could be properly implemented to alleviate the recurring, damaging attacks MS patients currently face without warning.

At the present time, there is no simple diagnostic test for MS. The diagnosis of MS currently remains primarily one of clinical evaluation. Since the time of Charcot's first observations, the definitive aspect of the diagnosis of MS has centered around the demonstration of lesions disseminated in time and space. In order to justify this diagnosis, a patient must either present with the occurrence of more than one attack (exacerbation/relapse) or progression of symptoms over many months, as well as, evidence of multiple discrete anatomical loci of disease in the white matter of the central nervous system (CNS) (Miller, 1998). Perhaps the most widely accepted diagnostic scheme was put forth by Schumacher and his colleagues in 1965. Patients are classified as having “clinically definite, probable, or possible” MS depending on the number of the following criteria described below which are applicable (Miller, 1998):

1. Age at onset between 10-50 years;

2. Objective neurological deficits present on examination referable to CNS dysfunction;

3. Neurological symptoms and signs indicative of CNS white matter disease;

4. Dissemination in time: two or more attacks (lasting at least 24 hours) and separated by at least one month (an attack is defined as the appearance of new symptoms or signs or worsening of previous ones) or the progression of symptoms and signs for at least six months;

5. Dissemination in space: two or more noncontiguous anatomical areas of brain involved;

6. No alternative clinical explanation.

The diagnosis of “clinically definite MS” requires that patients meet five or six of the criteria, always including the last one. Patients who fulfill fewer than five of the criteria, but always including the last, are diagnosed with either “clinically probable MS” or “clinically possible MS”.

For the purposes of demonstrating this invention, the bodily fluids collected and analyzed will include, but are not limited to, blood and blood products. Through a battery of optimization experiments, an enzyme-linked immunosorbent assay (ELISA) to measure MBP autoantibodies in the blood of MS patients and those not yet diagnosed has been developed. Precision studies have shown that the assay is precise and repeatable at both high and low antibody titers. Analytic recovery experiments indicate that normal plasma constituents do not interfere with the accurate detection of MBP autoantibodies using this ELISA and that both serum and plasma are suitable matrices for their measurement. Hemolyzed, icteric, and lipemic blood samples do not interfere in the assay and circulating concentrations of several common MS drugs do not inhibit the detection of MBP autoantibodies. The developed assay is simple, inexpensive, and rapid (2 hours).

More specifically, the method of the subject invention involves an ELISA which comprises: (1) mixing a sample of body fluid, particularly blood or a blood product, from a mammal, usually a human, with at least one compound effective to optimize the signal to noise ratio (the compound exemplified herein is heparin); (2) contacting the sample with an immunosorbent coated with at least one protein associated with MS (exemplified herein is myelin basic protein) having a high specific activity for at least one autoantibody (anti-MBP IgG or IgM); and, (3) determining an amount of at least one autoantibody bound by the protein or proteins on the immunosorbent using an antibody composition having an affinity for the autoantibody, illustrated by, but not limited to, purified anti-human IgG/IgM conjugated to horseradish peroxidase (HRP) that is operably linked to a signal generating system, illustrated by, but not limited to, tetramethylbenzidine (TMB) substrate.

Accordingly, it is an objective of the instant invention to provide a clinically sensitive and reliable immunoassay to measure autoantibodies, specifically to MBP, in the blood of MS patients.

It is a further objective of the instant invention to provide a less invasive and less costly alternative to current techniques.

It is a further objective of the instant invention to provide a rapid method of detection of MS by an immunoassay.

It is yet another objective of the instant invention to provide a simple, definitive test for diagnosis of MS.

It is a still further objective of the invention to supply a means of determining disease sub-classification and relative disease activity as determined by routine patient monitoring.

It is yet another objective of the instant invention to provide a diagnostic assay device for use in the method.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 illustrates the potential mechanisms for demyelination within the blood-brain barrier;

FIG. 2 shows the MBP autoantibody IgM levels in normal vs. clinically definite MS patients, wherein levels in MS patients are generally elevated (+2SD above normal levels);

FIG. 3 shows the results of myelin basic protein (MBP) autoantibody IgG levels in clinically stratified MS stable vs. relapse patients, wherein relapse patients exhibit more elevated levels;

FIG. 4 demonstrates the types of disease course in multiple sclerosis (MS), which are relapse-remitting, secondary-progressive, and primary-progressive;

FIG. 5 illustrates the ELISA protocol;

FIG. 6 illustrates the limited sensitivity of a Western blot;

FIG. 7 shows the results of MBP autoantibody IgG levels in normal vs. clinically definite MS patients, wherein levels in MS patients are generally elevated (+2SD above normal levels);

FIG. 8 demonstrates Receiver Operating Curves (ROC);

FIG. 9 demonstrates the diagnostic value of the developed ELISA;

FIG. 10 demonstrates the added sensitivity when markers are utilized in combination.

### DETAILED DESCRIPTION OF THE INVENTION

The purpose of this study was to develop an immunoassay to measure MBP autoantibodies in the blood of MS patients, thereby avoiding the invasive technique of lumbar puncture required for CSF collection. By examining matrix effects within the developed assay, both serum and plasma samples may be used. In addition, different autoantibody classes, that is IgG and IgM, may better indicate the disease status of the individual patient. However, other body fluids may apply which require minimally invasive techniques to obtain, such as saliva, and are contemplated by the present invention.

Given the invasive nature of lumbar puncture, its unsuitability as a widely accepted diagnostic measure and since the majority of these other markers have been measured in the CSF, clinical sensitivity and specificity of MBP autoantibodies and other markers must be re-confirmed in the blood of MS patients. The diverse clinical manifestations of MS, variations in immunological evidence of pathology between patients, and the presence of immune responses to different autoantigens suggests not only that MS is a heterogeneous disease but also emphasizes that accurate diagnosis with high sensitivity and specificity may mandate that multiple markers be incorporated into one test for the definitive diagnosis and progressive monitoring of the disease.

Due to the highly cationic nature of MBP, its charge may be the contributing factor for high background in a common enzyme-linked immunosorbent assay (ELISA). Non-specific charge interactions typically plague the sensitivities of ELISAs using positively charged proteins (Pesce et al., 1986). This obstacle was recognized early during the development of the disclosed ELISA protocol.

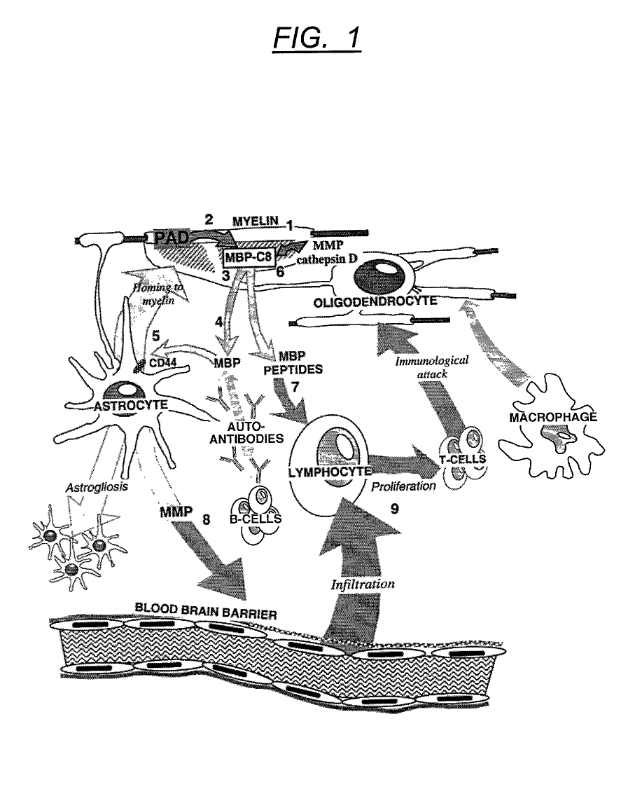

In the determination of the assay's clinical utility, sera/plasma of clinically definite MS patients should exhibit elevated titers of MBP autoantibodies when compared with titers in healthy controls, therefore offering a high clinical sensitivity and specificity. If MBP autoantibodies are involved in the pathogenesis of demyelinating lesions (FIG. 1), we expect that their levels should increase during periods of active demyelination, resulting in relapse or exacerbation of clinical signs in MS patients. Referring to FIG. 1, which is an excerpt from the Doctoral Thesis of Baldwin Mak, University of Toronto, 1999, demyelination begins with the disruption of the bilayer 1. Deimination of MBP by peptidylarginine deiminase (PAD) 2 and further disorganization of the bilayer 3 then occurs. This results in the shedding of MBP 4 and subsequent activation of astrocytes and induction of astrogliosis and homing to myelin 5. As the pathway continues, digestion of MBP by cathepsin D and matrix metalloproteinase (MMP) generates MBP peptides 6. Lymphocytes become sensitized 7 and MMP digests the BBB, thus allowing sensitized lymphocytes to cross the barrier 8. The autoimmune phase has commenced 9. The diagnostic utility of the developed MBP autoantibody assay will be compared with the clinical utility of assays measuring other biochemical markers for MS.

Measurement of plasma MBP autoantibodies (IgG) by enzyme-linked immunosorbent assay (ELISA) is effective in achieving the clinical objectives of high sensitivity (77%) and specificity (95%). Circulating anti-MBP IgG in MS appears to be an indicator of disease activity as levels were increased in patients who were experiencing relapse. The measurement of MBP autoantibodies by ELISA was also shown to be a more sensitive technique than Western blotting and appears to have the highest opportunity for clinical relevance compared with the measurement of autoantibodies to PLP, NSE, and S-100B alone, or presence of the proteins themselves.

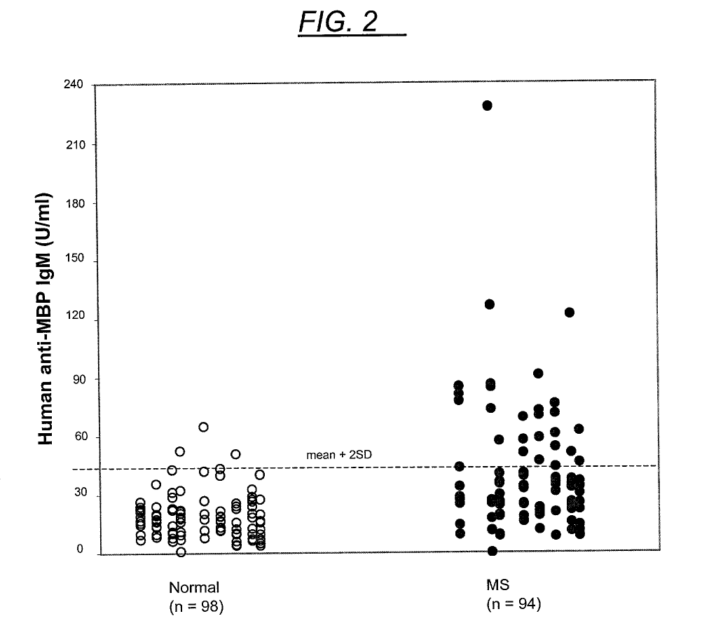

With respect to anti-MBP IgM levels, only 30% of the MS patients exhibited circulating elevated titers, as measured by ELISA (FIG. 2). Although one patient with an extremely elevated anti-MBP IgM level was experiencing a first relapse in MS, no significant difference was found between relapsing and stable patients on the whole. In fact, no definitive feature of the patients with elevated anti-MBP IgM could be ascertained. Elevated IgM in the CSF during episodes of relapse has been previously established in the literature, however, little focus has been devoted to determining the antigenic characterization of this IgM response. In one group of RRMS patients, MBP-specific IgM responses correlated well with CSF IgM indices (Annuziata et al., 1997). Furthermore, patients with high IgM anti-MBP suffered from fewer attacks, with a less rapid frequency and exhibited a decrease in EDSS score more readily as compared with low IgM anti-MBP patients. It appears that anti-MBP IgM primary response is the product of specific antigenic stimulation. Patients with high IgM followed a more favorable/benign course during follow-up. Therefore, long-term monitoring of this type of patient including duration and time between attacks, may be useful in the determination of the significance of elevated anti-MBP IgM in the circulation.

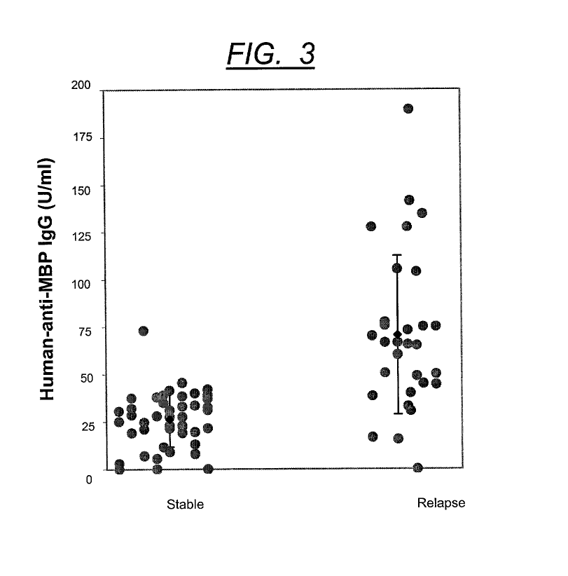

Levels of IgG in the stratified patient cohort were significantly elevated in the relapsing patients versus patients in a stable/remitting phase of their disease (FIG. 3). Thus, it appears that circulating anti-MBP IgG is indicative of disease activity. This observation was also reported by numerous investigators who measured anti-MBP IgG in the CSF. The nature of the disease dictates that relapses have various frequencies of occurrence, ranging from a couple months to several years. Therefore, MBP autoantibodies may be reliably detected in the circulation of MS patients. Moreover, the IgG class of antibodies may be a better indicator of disease activity than IgM. However, IgM may prove useful as an indicator of an initial episode or in predicting disease progression.

In order to further evaluate the specificity of the autoantibody response to MBP in MS patients, other autoantibody responses to both myelin proteins, as well as, non-myelin CNS-specific proteins were examined. Since MBP autoantibodies were the most elevated in the MS patients tested, as compared with PLP, NSE, or S-100B autoantibodies, the autoantibody response in MS appears to be specific for myelin basic protein. The 6% of patients who presented elevated anti-PLP IgG all exhibited elevated titers of MBP autoantibodies as well.

Analysis of autoantibodies for all three proteins (PLP, NSE, and S-100B) by Student's t-test revealed that there was no significant difference in the levels between normal and MS patients. ELISA was performed for proteolipid protein (PLP), which accounts for 50% of the total myelin protein, neuron specific enolase (NSE), a neuronal marker, and S-100B, an astrocyte marker.

In general, patients with autoimmune diseases tend to be reactive to multiple antigens. In order to accurately validate the diagnostic potential of the MBP autoantibody assay, it is critical to demonstrate that the antibody response to MBP was specific for MS patients. Although the autoantibody response in MS appears to be specific for myelin basic protein, the level of autoantibodies specific for NSE may prove beneficial as an indicator for axonal degeneration secondary to demyelination and the level of S100B may indicate the extent of gliosis or astrocyte/oligodendrocyte breakdown in certain MS patients.

It became apparent that the addition of a polyanion was required to aid in the charge neutralization of the MBP. After optimization experiments were performed, heparin was chosen because it significantly improved the distinction between control and MS patients while maintaining an excellent signal to noise ratio with the positive control. The sensitivity of the ELISA largely depends on the reduction of non-specific binding by other serum factors.

As a heterogeneous disease, clinical presentation and neuropathology are diverse between patients (Laman et al, 1998). Recent recommendations for the development of novel diagnostic measures in MS incorporate the fact that MS is a disease mediated immunologically; markers may be identified more easily in accessible peripheral fluids, i.e. blood, urine and CSF (Laman et al., 1998). Such a marker should meet acceptable assay performance requirements and correlate with disease stage.

Based on the evidence, should MBP autoantibodies in the blood of MS patients mimic the diagnostic benefit of MRI and identify possible MS patients as experiencing disease, whether used alone or in conjunction with MBP or Tm concentrations, MBP autoantibody measurement would offer clinicians a rapid, sensitive, relatively non-invasive, and inexpensive tool for the diagnosis and monitoring of the progression of MS.

The overall efficiency of a fully developed immunoassay is determined by the proportion of all patients for whom the test correctly predicts the presence or absence of disease (Micallef and Ahsan, 1994). The clinical utility of MBP autoantibody measurement was examined by using the developed ELISA to measure anti-MBP IgG and IgM in clinically definite MS patients. The present invention is based on the hypothesis that MBP IgG should correlate with disease activity. In addition, detection by Western blot, which has been employed by other investigators, of anti-MBP IgG in the sera of MS patients should demonstrate less sensitivity in comparison. Moreover, if the autoimmune response in MS is specific to MBP, autoantibodies to other myelin and non-myelin proteins should be negligible in comparison.

By “sample”, what is meant is a volume of body fluid (preferably obtained in a non-invasive manner), such as blood or blood products, saliva, or any body fluid from which a meaningful analysis may be performed, which is obtained at one point in time. Further, all the markers can be measured with one assay device or by using a separate assay device for each marker in which case aliquots of the same fluid sample can be used or different fluid samples can be used. It is apparent that the analyses should be carried out within some short time frame after the sample is taken, e.g., within about one-half hour, so the data can be used to prescribe treatment as quickly as possible. It is preferred to measure each of the markers in the same single sample, irrespective of whether the analyses are carried out in a single analytical device or in separate such devices so the level of each marker is measured simultaneously and the resulting presence in a single sample can be used to provide meaningful data.

As used herein the term “marker”, “biomolecule”, “biochemical marker”, or “marker protein”, refers to any enzyme, DNA, RNA, carbohydrate, steroid, lipid, protein, polypeptide, peptide, isomeric form thereof, immunologically detectable fragments thereof, or other molecule that is released from the brain during the course of MS pathogenesis. Such markers include, but are not limited to, any unique proteins or isoforms thereof that are particularly associated with the brain and/or proteins or isoforms thereof that are found in tissues other than the brain.

By “immunologically detectable” is meant that a marker or protein or fragments thereof contain an epitope which is specifically recognized by a cognate antibody or antibody reagents used in the assay.

The term “diagnostic” is meant that a marker or protein or fragments thereof is present at statistically significant levels indicative of initiation of disease state or state of disease activity, e.g. relapsing or progressing of disease activity.

The term “monitor” is used herein to determine the occurrence, to distinguish type, to measure severity, or to conclusively track progression of disease. In addition, the present invention relates to the usefulness of continued monitoring of MS patients for a period of time. This type of assessment could be very useful in the proper treatment of persons suffering from MS.

The terms “above normal” and “upregulated” are used herein to refer to a level of a marker that is greater than the level of the marker observed in normal individuals, that is, individuals who are not undergoing disease activity related to MS. For some markers, no or infinitesimally low levels of the marker may be present normally in an individual's body fluid, such as blood. For other markers analyzed, according to the invention, detectable levels may be present normally in a body fluid. Thus, these terms contemplate a level that is statistically significant or significantly above the normal level found in individuals.

The term “statistically significant” or “significantly” refers to statistical significance and generally means a two standard deviation (2SD) above normal, or higher, concentration of the marker. The assay method by which the analysis for any marker protein is carried out must be sensitive to be able to detect the level of the marker which is present over the concentration range of interest and also must be highly specific.

One embodiment is a method of diagnosing or monitoring MS in a mammal, preferably a human. A sample body fluid is obtained from the mammal, wherein in a preferred embodiment the body fluid is blood or blood products, e.g. serum, plasma and the like. The sample is contacted with at least one protein associated with multiple sclerosis, by way of an ELISA, and a level of at least one autoantibody specific for at least one protein in the sample is determined. The levels are compared with the level of the at least one autoantibody with statistically significant levels thereof, wherein diagnosis or monitoring of MS is achieved. In a preferred embodiment, the protein is MBP and the autoantibody is anti-MBP IgG, anti-MBP IgM, or both.

In a further embodiment, a kit may be assembled for diagnosing MS or monitoring disease state in MS patients, which comprises at least one biomolecule or an immunologically detectable fragment thereof which is upregulated in MS patients and being immobilizable on a solid support, where the biomolecule or biomolecules has an affinity for at least one additional biomolecule whose presence is diagnostic of MS. At least one labeled biomolecule having a binding affinity for the at least one additional biomolecule would be included; whereby the performance of at least one analysis determinative of the presence of statistically significant levels of the biomolecule or biomolecules or an immunologically detectable fragment thereof, is carried out on a sample of body fluid and provides a means for diagnosing or monitoring disease state. Preferably, a biomolecule would be represented by MBP whereby an additional biomolecule would be anti-MBP IgG, anti-MBP IgM, or both. The labeled biomolecule would preferably be antihuman IgG/IgM (depending on the choice of additional biomolecule) conjugated to HRP.

All samples would be taken at the same time or at different time periods with monitoring of disease activity generally beneficial with one or more samples obtained at various times during disease course.

Disclosed herein is a unique ELISA procedure for detection of MS-specific autoantibodies. The following examples are given for the purpose of illustrating various embodiments of the invention and are not meant to limit the present invention in any fashion.

### EXAMPLE 1

Materials and Methods

MS patient sample size was determined by currently available patients. Under the assumption of a conservative estimate of a two standard deviation (2SD) difference between MS patients and controls, the minimum sample size of 10 patients provides 80% power to detect a type I error at p=0.01.

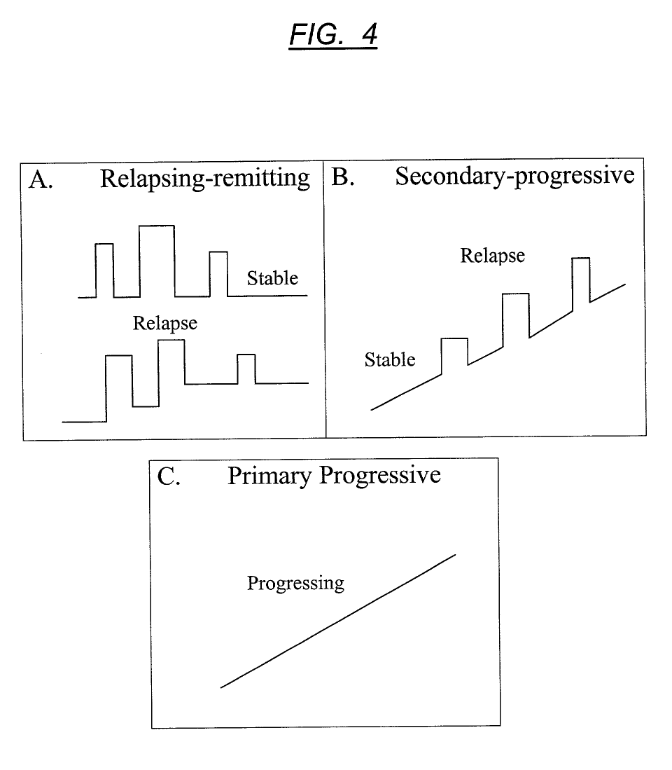

Consecutive MS patients meeting the diagnosis of clinically definite MS, as described by Poser et al. (1983), seen in consult at the St. Michael's Hospital (SMH), MS Clinic, Toronto, Ontario, were offered study inclusion. Baseline assessments were made in the clinic including date of birth (DOB), sex, date of blood collection, when available: date of symptom onset and date of diagnosis, as well as a detailed neurologic examination, determination of disease severity score (EDSS; Expanded Disability Scaled Score) and categorization of current disease status (FIG. 4) (active relapse, remission, primary-progressive, secondary-progressive).

Blood samples from ninety eight apparently healthy individuals, with no previous symptoms of MS, were drawn at Syn X Pharma, Inc. DOB and gender were recorded at the time of blood collection and a general health questionnaire was used to record any other diseases in the individual donors.

Following patient consent, phlebotomy was performed on ninety-six MS patients. Each patient was assigned an SMH number at the time of blood collection to ensure the confidentiality of the patients and provide anonymous analysis of the blood until clinical utility of the test was assessed. The blood was collected into matched heparinized and serum Vacutainer tubes (Beckton-Dickenson) and centrifuged at 3,000 rpm for 30 minutes. Each patient's plasma and serum fractions were aliquoted and frozen at −20° C. until use. The patient information was obtained from the clinic charts after analysis by ELISA.

### EXAMPLE 2

MBP Autoantibody ELISA

Microtiter Plate Preparation

All odd column wells of 96 well microtiter plates (MaxiSorp, Nunc) were coated with 125 mL of 8 mg/L unfractionated myelin basic protein (bovine) in 100 mM carbonate/bicarbonate buffer, pH 9.6. All even column wells were coated with 125 mL of 100 mM carbonate/bicarbonate buffer (modified Crimando and Hoffman, 1992). Plates were sealed with ELISA plate sealer (Costar) and incubated at 4° C. overnight. Plates were then washed three times with 10 mM PBS+0.05% Tween-20, 300 mL/well. All wells were subsequently blocked with 250 mL/well of 2% w/v BSA (Equitech-Bio) in carbonate/bicarbonate buffer, to reduce non-specific binding, and were incubated at 4° C. overnight.

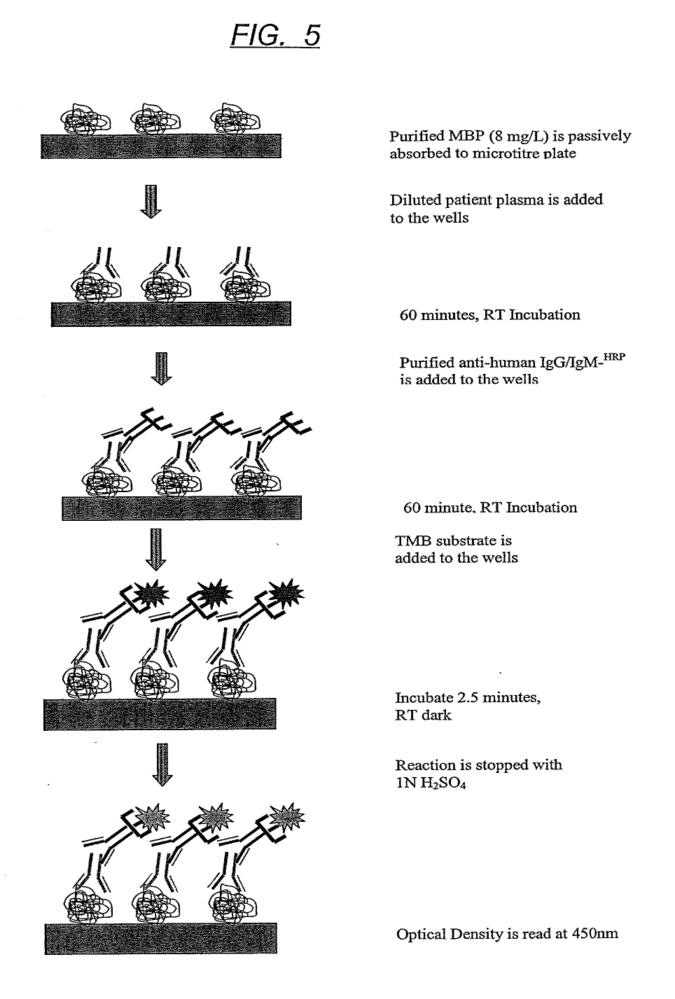

ELISA Protocol (FIG. 5)

Plates were washed three times with 10 mM PBS+0.05% Tween-20. Plasma test samples were diluted in the dilution buffer which consisted of 10 mM PBS, 0.05% Tween-20 and 5 USP/mL heparin (Sigma) (Pesce et al., 1986) at a working dilution of 1:320. Added to the matched MBP and adjacent MBP-free wells was 100 μL of each diluted test sample. Affinity purified goat polyclonal antibody against bovine unfractionated MBP was diluted in a calibrant of normal human plasma, diluted at 1:320 in the dilution buffer, and tested at 0, 25, 50, 100 and 200 ng/mL to form a standard curve. Negative controls were run in quadruplicate and consisted of neat dilution buffer. The wells were incubated for 1 hour at room temperature. Plates were then washed three times with 10 mM PBS+0.05% Tween-20. Wells containing test samples and the negative control were incubated with 100 μL/well of goat anti-human IgG (Fc) conjugated to horseradish peroxidase (Jackson) or goat anti-human IgM conjugated to horseradish peroxidase (Jackson), diluted 1:15,000 or 1:20,000 respectively, in dilution buffer for 1 hour at room temperature. The wells containing the goat polyclonal antibody were incubated with 100 μL of donkey anti-goat IgG conjugated to horseradish peroxidase (Jackson), diluted 1:7,000 in dilution buffer for 1 hour at room temperature. After three washes with 10 mM PBS+0.05% Tween-20, 100 mL of tetramethylbenzidine (TMB) (Moss) was added to each well and incubated in the dark for 2.5 minutes. The reaction was stopped with 1 N H₂SO₄, 100 μL/well. Optical density was read on a SoftMax microtiter plate reader (Molecular Devices) at 450 nm. Absorbance values from the non-coated well were subtracted from the OD 450 nm value in the MBP coated well from the same sample. This subtracted value (S-value) reflects the specific antibodies present in the sample. The subtracted values obtained from the standard curve have been used to quantitate the amount of IgG in each test sample.

### EXAMPLE 3

Western Blot Protocol for the Detection of MBP Autoantibodies IgG

The equivalent of 2 μg unfractionated myelin basic protein (bovine) per lane was separated by 12.5% sodium dodecyl sulfate polyacrylamide gel electrophoresis (SDS-PAGE) at 180 volts(V) for 60 minutes at room temperature (Laemmli, 1970) as well as, 6 mL of precision molecular weight markers (BIORAD). The gels were then transferred to Immobilon P PVDF membranes (Millipore) at 100 V for 180 minutes at 4° C. using 10% methanol in transfer buffer. Following electroblotting, the membranes were blocked with 5% Blotto/TBS and allowed to incubate for 60 minutes, shaking at room temperature and then incubated at 4° C. overnight. The membranes were washed for 30 minutes at room temperature, shaking with Tris buffered saline+Tween-20 (TTBS). The membranes were secured in the MiniCell Protean III multi-sample apparatus. Thirty-eight normal serum samples and forty-two clinically definite MS samples were randomly chosen and diluted 1:10 in 1% Blotto/TBS. Diluted serum samples (240 mL of each sample) were loaded into separate lanes on the apparatus. The diluted serum samples were incubated for 60 minutes, shaking at room temperature and all sera was subsequently aspirated off the membrane using a vacuum pump. The membranes were washed three times, each for 5 minutes with TTBS. Goat anti-human IgG (Fc) conjugated to horseradish peroxidase (Jackson) was diluted 1:2,000 in 1% Blotto/TTBS. Membranes were incubated with the conjugated antibody for 120 minutes, shaking at room temperature. The membranes were subsequently washed three times, each for 5 minutes, shaking at room temperature with TTBS. The immunoblots were developed using a TMB substrate kit as per the manufacturer's instructions for 3 minutes. The reaction was stopped with ultra-filtered water.

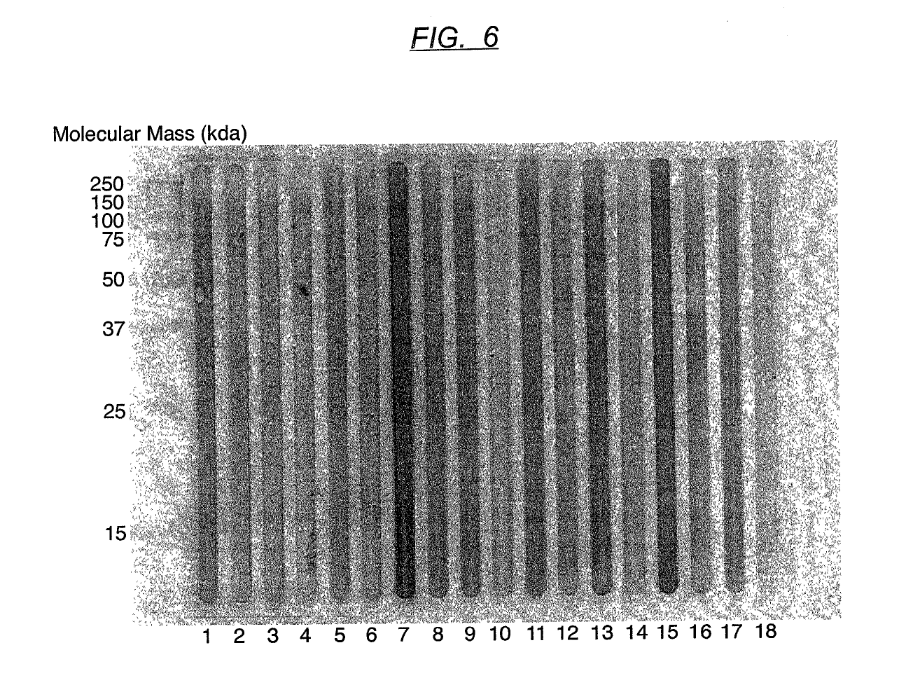

In order to evaluate Western blot analysis as a secondary method of MBP autoantibody detection, normal and clinically definite MS patient samples were analyzed for MBP specific IgG (FIG. 6). Sensitivity measurement at low titers of MBP autoantibodies in the circulation and confirmation of sensitivity regarding an ELISA protocol of the instant invention were focused upon. None of the MS patient samples demonstrated a definitive band at 18.5 kDa, corresponding to MBP. This may be explained by the fact that the variation in background for the MS patients tested by Western blot can not be adjusted for as is done in the ELISA format, by the addition of heparin (Alcantara et al., 1999) and the subtraction of an individual sample background. This merely compounds the difficulty in achieving reasonable sensitivity by this method.

### EXAMPLE 4

MBP Autoantibody ELISA Development And Assay Performance

Numerous optimization experiments must be conducted during the development of an immunoassay in order to maximize analytic and clinical sensitivity, as well as minimize non-specific interference within the assay. Since most assays for specific antibodies are qualitative, these experiments generate an assay which minimizes false positive results and allow for a low detection limit required to detect low titer antibody levels (Micallef and Ahsan, 1994), while maintaining low levels of imprecision. Upon the completion of the fully optimized assay, experiments to determine performance characteristics are conducted to show that the assay is precise and repeatable. For example, MaxiSorp C plates offered a more reliable measurement of antibody while maintaining an excellent signal to noise ratio. Therefore, MaxiSorp microtiter plates were the optimal solid phase for this assay.

### EXAMPLE 5

Dilution Factor Determination

The optimal serum/plasma dilution factor was chosen by running eleven normal samples and five clinical samples in the anti-MBP IgG ELISA. The choice of 1:320 as the plasma dilution was based on the fact that the point of inflection for the majority of the normal samples in their serial dilution was observed at this dilution factor.

As previously mentioned MBP is a cationic protein and therefore non-specific charge interactions were hypothesized to contribute to the lack of distinction between normal and MS patient MBP autoantibody titers. Heparin (5 USP) was placed in the plasma dilution buffer due to the success with the polyclonal control (rabbit). At the optimal dilution factor of 1:320, only 2 of the initial 24 MS patient samples exhibit antibody titers elevated above the mean +2SD of the normal range (n=11). Clinical detection, via sensitivity, dramatically improved when the antibody dilution buffer was modified to include heparin. This affords a possible reason as to why some investigators achieved successful detection of MBP autoantibodies where others had previously failed. Upon examination of those reports denying the presence of anti-MBP, the assay protocols employed simple buffers, which did not appear to be specific for such a cationic protein.

Plasma from two separate MS patient samples was added to plasma from each of three normal donors and were assayed for anti-MBP IgG. The S-value for both MS patient samples was within a 5% margin of recovery. Traditionally, analytic recovery experiments are performed by adding a known concentration of purified analyte (in this case purified antiMBP IgG) into normal samples. However, this technique can not be performed for this assay since purified MBP IgG is not commercially available. Nevertheless, this recovery experiment illustrates that MBP autoantibody titers are not subject to interference by normal plasma constituents.

The effect of interfering substances such as hemoglobin, lipids, or bile acids are generally appreciated by the inclusion of hemolyzed, lipemic, or icteric blood in the normal cohort of samples examined (Miller and Levinson, 1996). However, it is important to test these possible interfering substances in the developed assay by adding known quantities of hemoglobin, lipid, and bilirubin into patient samples. Moreover, the extent to which specific pharmacotherapeutic drugs for the disease of interest interfere with the testing of the analyte is also critical. By evaluating patient samples containing added increasing quantities of common therapeutics, the extent of possible drug interference can be determined.

In order to assess whether standard interference compounds, such as hemoglobin, bilirubin, and lipid prevent the accurate measurement of MBP autoantibodies, three concentrations each of hemoglobin, bilirubin, and triglyceride were added to the plasma from one MS patient. Each sample was assayed for MBP IgG in duplicate. When compared with the subtracted OD values (S-values) achieved by the control samples (MS patient plasma without any additional compounds), samples with elevated hemoglobin, bilirubin, and triglyceride yielded similar OD values. The three concentrations of each compound tested, represent values outside the normal range, as well as within the normal range. Therefore, MS patients who present with hemolyzed, icteric, or lipemic blood samples can still be reliably tested for MBP autoantibodies by this assay.

Patients with autoimmune disease generally attempt to control their symptoms with some form of agent. Many patients prefer such pharmaceutical agents as methylprednisolone (SOLU-MEDROL), IFNβ-1a (REBIF/AVONEX), and glatiramer acetate (COPAXONE). Interferon β (IFNβ)-1b (BETASERON), and IFNβ-1a (REBIF/AVONEX) are both considered effective “first line” treatments in RRMS, in that they reduce relapses and MRI lesions, both indicators of disease activity in RRMS.

Although the mechanism of action has not been clearly defined for the β-interferons, their effects are likely that of immunomodulation because β-interferons are anti-inflammatory cytokines. They may also have significant capability of slowing down lymphocytic trafficking across the BBB, a theory suggested because they tend to suppress the development of gadolinium (Gd)-enhancing MRI lesions.

Glatiramer acetate (COPAXONE) is a linear polymer of the L-amino acids: glutamate, tyrosine, alanine, and lysine, which comprise an immunodominant region of myelin basic protein. COPAXONE'S efficacy lies in its ability to reduce the number of relapses in the individual patient. COPAXONE is thought to downregulate immune responses directed against myelin components by stimulating the generation of suppressor cells that are capable of reducing immune response by release of anti-inflammatory cytokines. Although this drug has been shown to reduce relapse rate and MRI lesion activity, COPAXONE has no effect on short-term disease progression as measured through the EDSS.

Methylprednisolone (SOLU-MEDROL) is a corticosteroid which is used to treat inflammation. SOLU-MEDROL is commonly administered to MS patients during acute relapse because it is deemed to have the effect of closing the damaged BBB and reducing inflammation in the CNS (van den Noort and Holland, 1999).

Paclitaxel (TAXOL) is a well known anti-cancer agent. Recent evidence (Cao et al., 2000) has shown that paclitaxel is an effective agent in attenuating the clinical manifestations of experimental allergic encephalomyelitis (EAE), the classic T-cell-mediated animal model of MS. TAXOL, as a valuable early treatment for MS, is contemplated by this invention.

In the assessment of possible interference of common MS pharmaceutical agents, increasing doses of SOLU-MEDROL, REBIF/AVONEX, COPAXONE and TAXOL were added to the diluted plasma of one MS patient. With the exception of SOLU-MEDROL, the remaining three drugs did not significantly decrease the endogenously detected titer of MBP autoantibodies. SOLU-MEDROL concentrations commencing with 375 mg/mL significantly inhibited the accurate detection of MBP autoantibodies in the patient's sample. The recommended dose for this drug ranges between 10-6,000 mg. Since 375 mg/mL translates to over 50 g in the average individual, the detection of MBP IgG in a patient receiving this treatment would remain unaffected. MBP IgG may be a useful biochemical marker for monitoring MS progression in patients undergoing novel therapies.

In order to quantitate the results achieved in this assay, a standard curve using affinity purified anti-MBP IgG, raised in goat, was obtained. Using three separate assay runs, the standard curve exhibits linearity from 0 to 100 U/mL. This standard curve has been used to quantitate the amount of IgG in each patient sample.

### EXAMPLE 6

MBP Autoantibody ELISA

Seventy-four MS patients were stratified on the basis of disease status at the time of blood collection, irrespective of disease course. As illustrated in FIG. 3, patients in relapse (n=26) had MBP IgG autoantibody levels significantly elevated (p<0.001, Student's t-test) compared with those patients in stable courses of the disease (n=48).

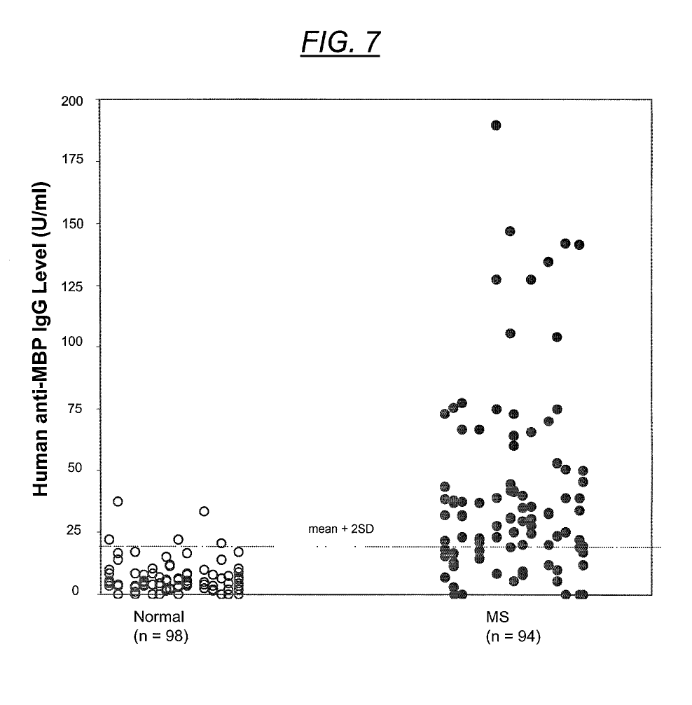

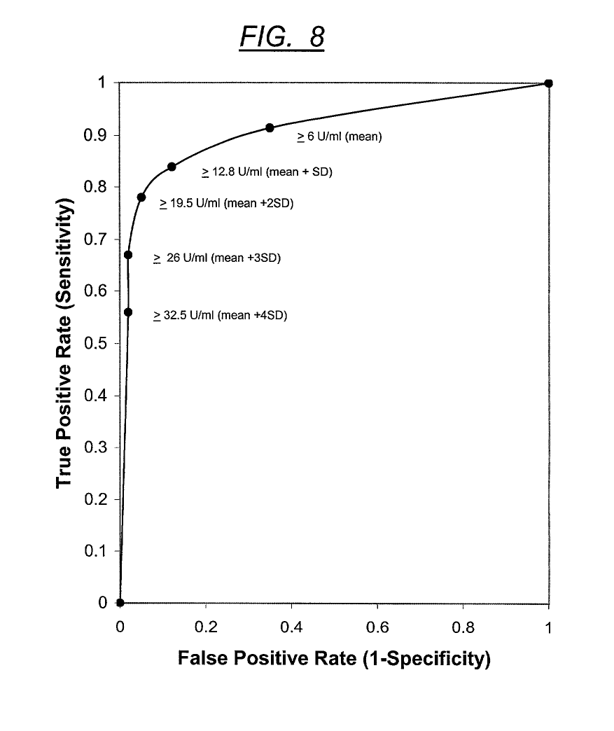

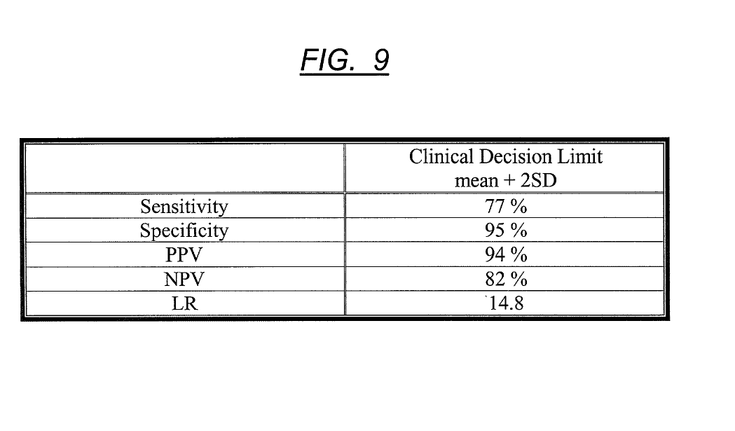

In a further experiment, ninety-eight normal healthy subjects (age range 20-66, mean age=36, approximately equal number of males and females) were run on the ELISA, in duplicate. Plasma samples from ninety-four clinically definite MS patients (age range 18-63, mean age=38, male and female) (FIG. 7) were also run on the ELISA. Using the mean +2SD of the normal subjects studied as the clinical cut-off (dashed line), 77% of the MS patients tested exhibited elevated levels of MBP autoantibodies (IgG) in plasma (p<0.001), whereas only five normal samples exhibited IgG levels marginally above clinical cut-off. Receiver Operating Curves (ROC) (FIG. 8) are constructed by plotting the sensitivity vs. the specificity of the test using multiple measurements as possible clinical decision limits. Determination of the optimal clinical cut-off by ROC plot illustrates that using the mean +2SD of the normal samples as the clinical decision limit offers high sensitivity (77%) and specificity (95%) (FIG. 9).

Sensitivity and specificity describe attributes of the test when the actual clinical diagnosis is known (i.e. the proportion of those with MS who actually have a positive test result or the proportion of those who are healthy who will have a negative test result). Since, in clinical practice one does not usually know who has the disease, positive predictive value (PPV) and negative predictive value (NPV) are useful in determining how likely a patient is to have the disease given a positive test result. PPV is the proportion of patients with a positive test result who have the disease. NPV is the proportion of patients with a negative test result who do not have the disease. Although useful, PPV and NPV vary according to the prevalence of the disease (i.e. the proportion of people undergoing the test who actually have the disease). Sensitivity and specificity are usually stable regardless of the prevalence of disease in the population in which the test is conducted while predictive values vary considerably in different populations of patients. Likelihood ratios (LR) permit a calculation of the probability of disease for a specific test result and specific disease prevalence. Likelihood ratios with a value greater than 10 are usually judged to be of high diagnostic value. This assay offers an LR of 14.8 and thus is a reliable diagnostic tool for MS.

### EXAMPLE 7

MBP, S-100B, NSE, and Tm ELISA

MBP, S-100B, NSE and Tm concentrations were measured in ninety matched MS patient serum samples, by two-site, in-direct ELISA as per the manufacturer's instructions for the Smart MBP kit, Smart S-100B kit, Smart NSE kit and Smart Tm kit (SynX Pharma, Inc.) respectively. The clinical decision limit was determined by the evaluation of 103 normal healthy donors by the manufacturer and was defined as the mean +2SD of the normal population tested. It should be noted that not all MS patient sample analysis could be included in this work, as some of the serum samples were hemolyzed and posed interference problems in the NSE assay.

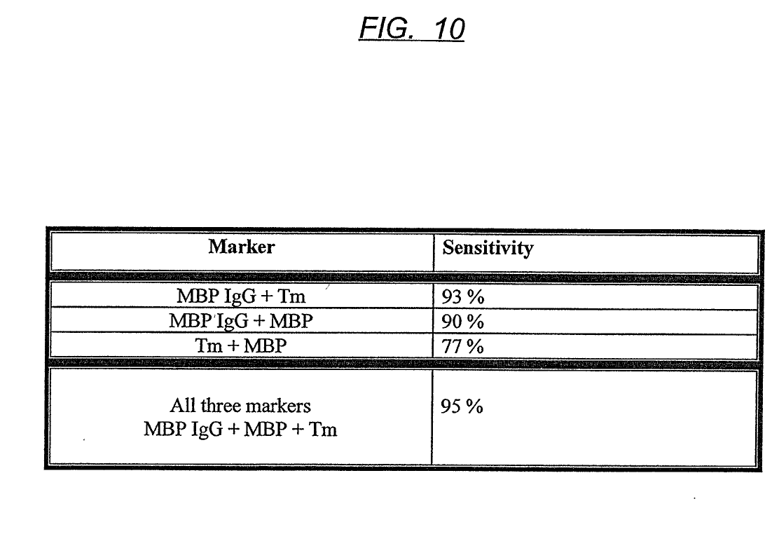

Serum concentrations of Tm, MBP, and NSE were shown to have clinical utility in MS. However, when MBP, Tm, and MBP autoantibody measurements are all considered in the diagnostic work-up of the MS patients, clinical sensitivity increases to 95% which can not be achieved by the measurement of any of these markers alone (FIG. 10). The adoption of a panel of markers may optimize the usefulness of each of these markers in the diagnosis of MS.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for diagnosing or monitoring multiple sclerosis (MS) in a mammal comprising:
obtaining a sample of body fluid from said mammal, wherein said body fluid includes blood, blood products and saliva;
contacting said sample with at least one protein associated with multiple sclerosis, wherein said contacting is by an enzyme-linked immunosorbent assay (ELISA);
determining a level of at least one autoantibody specific for said at least one protein in said sample; and,
comparing said level of said at least one autoantibody with statistically significant levels thereof, wherein diagnosis or monitoring of MS in said mammal is achieved.

2. The method of claim 1, wherein said mammal is a human.

3. The method of claim 1, wherein said protein is myelin basic protein (MBP).

4. The method of claim 1, wherein said ELISA comprises the steps of:
mixing said sample with at least one compound effective to optimize the signal to noise ratio;
contacting said sample with an immunosorbent comprising said at least one protein having a high specific affinity for said at least one autoantibody; and,
determining an amount of said at least one autoantibody bound by said at least one protein on said immunosorbent using an antibody composition having an affinity for said at least one autoantibody and operably linked to a signal generating system.

5. The method as in claim 4, wherein said signal generating system is a tetramethylbenzidine substrate.

6. The method as in claim 4, wherein said at least one autoantibody is anti-MBP IgG.

7. The method as in claim 6, wherein said antibody composition comprises purified anti-human IgG conjugated to horseradish peroxidase.

8. The method as in claim 4, wherein said at least one autoantibody is anti-MBP IgM.

9. The method as in claim 8, wherein said antibody composition comprises purified anti-human IgM conjugated to horseradish peroxidase.

10. The method as in claim 4, wherein said at least one autoantibody includes anti-MBP IgG and anti-MBP IgM.

11. A kit for diagnosing multiple sclerosis (MS) or monitoring disease state in MS patients, comprising:
at least one biomolecule or an immunologically detectable fragment thereof which is upregulated in MS patients, said biomolecule having an affinity for at least one additional biomolecule whose presence is diagnostic of MS, said at least one biomolecule being immobilizable on a solid support; and,
at least one labeled biomolecule having a binding affinity for said at least one additional biomolecule;
whereby performance of at least one analysis determinative of the presence of statistically significant levels of said at least one biomolecule or an immunologically detectable fragment thereof, is carried out on a sample of body fluid and provides a means for diagnosing or monitoring disease state.

12. The kit as defined in claim 11, wherein said sample of body fluid is blood, blood products, or saliva.

13. The kit as defined in claim 11, wherein said at least one biomolecule is myelin basic protein (MBP).

14. The kit of claim 11, wherein said at least one additional biomolecule includes anti-MBP IgM and anti-MBP IgG.

15. The kit as defined in claim 11, wherein said at least one additional biomolecule is anti-MBP IgM.

16. The kit as defined in claim 15, wherein said labeled biomolecule is anti-human IgM conjugated to horseradish peroxidase.

17. The kit as defined in claim 11, wherein said at least one additional biomolecule is anti-MBP IgG.

18. The kit as defined in claim 17, wherein said labeled biomolecule is anti-human IgG conjugated to horseradish peroxidase.

19. The kit of claim 11, wherein said monitoring is carried out on a single sample.

20. The kit of claim 11, wherein said monitoring is carried out on multiple samples such that at least one analysis is carried out on a first sample and at least another analysis is carried out on a second sample.

21. The kit of claim 20, wherein said first and second samples are obtained at different time periods.
